(19) United States
(12) Patent Application Publication
(10) Pub. No.: US 2005/0065471 A1
(43) Pub. Date: Mar. 24, 2005

(54) Continuous safe suction device

(76) Inventor: Charles Kuntz, Highett Vic (AU)

Correspondence Address: Richard C. Litman;LITMAN LAW OFFICES, LTD., P.O. BOX 15035, Arlington, VA, 22215, US

(21) Appl. No.: 10/929,790

(22) Filed: Aug. 31, 2004

Related U.S. Application Data
(60) Provisional application No. 60/504,989, filed on Sep. 23, 2003.

(51) Int. Cl. A61M 37/00

(52) U.S. Cl. 604/133

(57) ABSTRACT

The continuous safe suction device safely removes fluids from a patient's body. The device has a housing for storing a suction-producing member. The suction-producing member is a collapsible bulb or, in another embodiment, a spring-loaded membrane and casing that when compressed, upon recoil will apply gentle suction to an input tube that drains fluid from the patient and transports it into the suction bulb or membrane and casing. The suction bulb or casing has a pair of one-way valves secured in-line with respective input and output tubes. Upon complete filling it is recompressed to force the fluid in the bulb out through the output tube, which transports the fluid to an exterior fluid collection bag. The suction bulb or casing is compressed by a screw piston, controlled by a sensor that determines when the bulb is filled and emptied, the screw piston, in one embodiment, being withdrawn incrementally.

### CROSS-REFERENCE TO RELATED APPLICATION

This application claims the benefit of U.S. Provisional Patent Application Ser. No. 60/504,989, filed Sep. 23, 2003.

### BACKGROUND OF THE INVENTION

1. Field of the Invention

The present invention relates to medical devices for draining fluids from a patient's body and, more particularly, to a continuous safe suction device using a suction bulb with one-way valves that prevent the drained fluid from flowing back to the patient.

2. Description of the Art

There are many existing medical devices currently used for draining fluid from a patient's body. Body fluids are often transferred through tubes from the body of the patient to an exterior collection container for testing and storage. A suction device is often used to draw the body fluids through the transfer tubes. Several body fluid drainage devices have been developed.

International Patent No. WO 90/07942, published on Jul. 26, 1990, discloses a method of continuous monitoring of the operation of a delivery system, a device for carrying out this method, and the use of this device. The device comprises a volumetric pump, an infusion control unit, an electronic measurement unit and an outlet catheter provided with a measuring element. The distinguishing feature of this method is that after each cycle of the pump, the actual pressure and/or volume of the delivery is measured in the catheter by measuring the amplitude of the electric signal coming from the measuring element. The measured signal is then compared with reference numbers in an analyzing system. The method and device of the '942 patent is preferably used for insulin administration.

U.S. Pat. No. 5,800,425, issued on Sep. 1, 1998 to DeLeonardis, discloses an automatic nasal aspirator for cleaning matter in the nasal cavity of an infant. The aspirator includes a collection member, a transfer tube and a suction device. The suction device causes suction so the matter in the nasal cavity is transferred through the tube into the collection

U.S. Pat. No. 5,405,319, issued on Apr. 11, 1995 to Abell et al., discloses a bowel evacuation system. The system comprises an elevated water bag with a check valve in the delivery line from the water bag into a rectal insertion tube, a drain line leading from the rectal insertion tube with a disposable valve member for opening and closing the drain line, and at least one pressurizing member in the form of manual squeeze bulbs that simultaneously open the check valve for delivery of water from the water bag through the rectal insertion tube.

U.S. Pat. No. 5,399,166, issued on Mar. 21, 1995 to Laing, discloses a portable infusion device. The device comprises a housing, a flexible air bag contained inside of the housing, a space next to the air bag for containing a drug bag, a line connected between the drug bag and the patient, a pump connected to the airbag, and a control unit for controlling the operation of the pump. The control unit includes a sensor for determining if the fluid pressure drops below a predetermined level.

U.S. Pat. No. 4,828,546, issued to McNeil et al. on May 9, 1989, discloses a bulb evacuator for closed wound pair of inlets with one-way valves disposed on a removable cap that covers an opening in the top of the compressible container. The opposite end of the bulb includes an integrally formed anti-reflux valve and a latching device. A separate collection bag may be secured to the latching device so that once the bulb evacuator is filled with fluid, it may be compressed so that the fluid is forced in to the collection bag.

U.S. Pat. No. 4,592,741, issued to Vincent, M. J. on Jun. 3, 1986, discloses a medical apparatus for the aspiration of pneumothorax. The device is used for aspirating an air pocket surrounding a collapsed lung. The device includes a needle that is inserted into the cavity around the collapsed lung. The needle is connected to a pump by a hose. A flow meter is provided to measure the airflow rate through the hose and the suction pump. A vent line is provided between the suction pump and the flow meter to prevent sudden increases in airflow rate.

U.S. Pat. No. 4,539,005, issued on Sep. 3, 1985 to Greenblatt, discloses a blood infusion apparatus. The apparatus provides a pneumatic bladder supported within a housing. A hinged door supports a unit bag containing fluid on the housing presses the unit bag against the pneumatic bladder, which is inflated by a foot pump opening of an evacuation valve provides instant deflation of the bladder, allowing rapid removal of the first unit bag and replacement with a full unit bag, and rapid re-inflation of the bladder and successive diffusion of unit bag fluids into the patient.

U.S. Pat. No. 4,416,658, issued to Numazawa et al. on Nov. 22, 1983, discloses a blood-suctioning device. The device is controlled by a pressure sensor. The sensor controls a roller pump that removes blood from a surgical area through a plurality of transfer tubes.

U.S. Pat. No. 4,392,858, issued to George et al. on Jul. 12, 1983, discloses a wound drainage device. The device includes a manually operated suction pump having inlet and outlet ports, and a pliable storage bag having an inlet. The pump inlet port is connected to catheter for removing drainage fluid from a wound. The pump outlet port is connected to the storage bag.

U.S. patent Publication No. 2003/0105437, published on Jun. 5, 2003 and applied for by Neubert, discloses an aspiration flow meter. A surgical pump includes a collection reservoir and and tissue from a surgical area. Aspiration tubing connects a surgical hand piece and the collection reservoir. A flow meter is electronically connected to the aspiration tube or the reservoir.

Currently used mechanical drainage devices are subject to failure and/or human error and often harm, and potentially cause death to, the patient. The patient is often harmed by draining fluid leaking back into the patient's body. Another problem with the conventional drainage devices is that they are bulky and need to be plugged into electrical outlets or centralized wall-mounted suction hoses. Also, most of the conventional fluid drainage systems include manual pumps. Finally, the conventional devices only allow monitoring of one patient at a time.

None of the above inventions and patents, taken either singly or in combination, is seen to describe the instant invention as claimed. Thus, a continuous safe suction device solving the aforementioned problems is desired.

### SUMMARY OF THE INVENTION

The continuous safe suction device is a device for safely removing fluids from the thorax or other parts of the body. The device has a housing for storing a suction producing member. The suction producing member is a collapsible bulb or membrane suction storage member that, when compressed, upon recoil will apply gentle suction to an input tube. The suction bulb has a pair of one-way valves secured to its top surface. Alternatively, one of the one-way valves may be positioned on the bottom of the suction bulb. An input tube is secured to one of the valves and an output tube is secured to the other valve.

The input tube drains fluid from the patient and transports it into the suction bulb. Once the bulb is completely filled it is recompressed to force the fluid in the bulb out through the output tube, which transports the fluid to an exterior fluid collection bag. The suction bulb is compressed by a screw piston, which is controlled by a sensor. The sensor determines when the bulb is filled to subsequently cause the screw piston to compress the bulb. The housing includes an integrated keyboard and read out screen for operating the suction device. In another embodiment a membrane suction storage member is employed in a similar manner with one-way valves in the input line and output line.

These and other features of the present invention will become readily apparent upon further review of the following specification and drawings.

### BRIEF DESCRIPTION OF THE DRAWINGS

FIG. 1 is an environmental, perspective view of a continuous safe suction device according to the present invention.

FIG. 2 is a perspective view of the continuous safe suction device.

FIG. 3 is a front view of the continuous safe suction device of the present invention with the housing broken away to show a portion of the interior.

FIG. 4 is a perspective view of an alternative embodiment of a continuous safe suction device according to the present invention.

FIG. 5 is a perspective view of the device of FIG. 4 with the front half of the case removed.

FIG. 6 is a front elevation view of the device of FIG. 4 with the front half of the case removed.

FIG. 7 is a rear view of the device of FIG. 4.

FIG. 8 is a section view of the diaphragm pump of the device of FIGS. 4-7.

FIG. 9 is an exploded perspective view of the device of FIG. 4.

FIG. 10 is a block diagram of a continuous safe suction device according to the present invention.

Similar reference characters denote corresponding features consistently throughout the attached drawings.

### DETAILED DESCRIPTION OF THE PREFERRED EMBODIMENTS

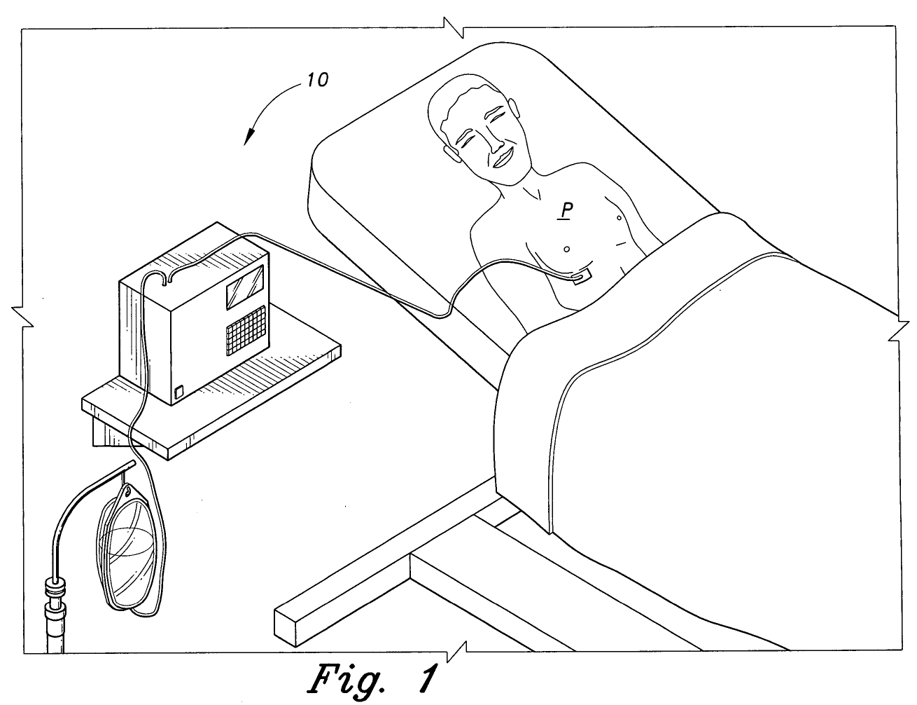

The present invention is a device for safely draining fluids from a patient and transferring the fluids to a collection member. The device includes an integrated computer that allows for continuous safe suction. FIG. 1 is an environmental, perspective view of the continuous safe suction device 10 being used on a patient P. The suction device 10 may be used with any form of drainage tube, but in the embodiment depicted in FIGS. 1-3 the suction device 10 is providing continuous safe suction to thoracic tubes. The suction device 10 is preferably positioned at a height lower than the patient P and the collection member is located below the suction device 10.

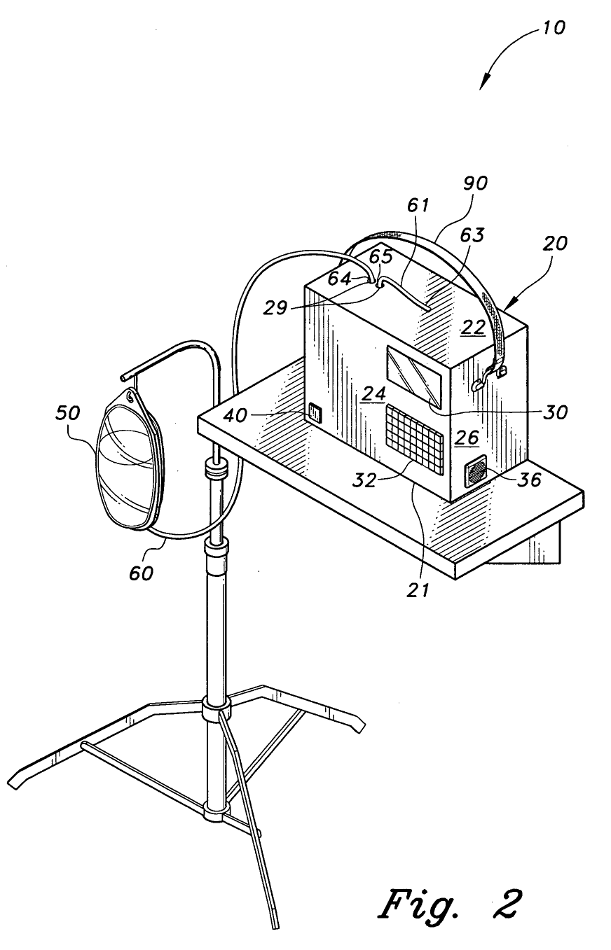

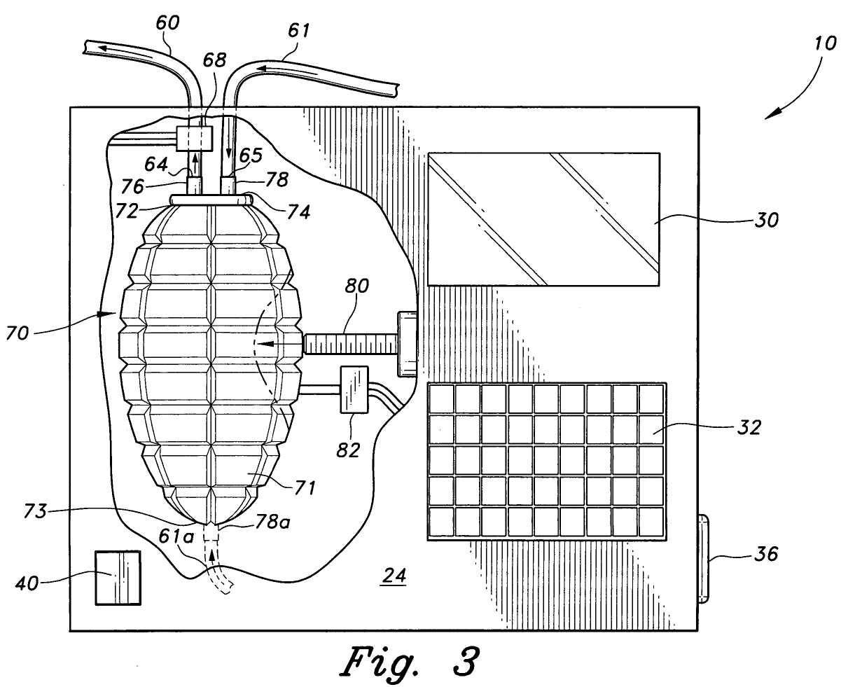

FIG. 2 is a perspective view of the continuous safe suction device 10. The suction device 10 generally includes a protective chamber or housing 20, a suction producing member 70 (shown in detail in FIG. 3), an input tube 61, an output tube 60, a fluid collection device 50 and a computer for controlling the suction device 10. The housing 20 comprises a top wall 22, a front wall 24, a pair of sidewalls 26, a bottom wall 21, and a rear wall (not shown). The housing 20 further includes access holes 29 disposed along the top wall 22 of the housing 20. The access holes 29 receive the input tube 61 and the output tube 60. One of the access holes 29 may optionally be disposed along the bottom wall 21 of the housing 20 to allow access through the bottom of the housing 20.

The suction device is controlled by an integrated computer of known construction and operation. The computer will record data from the suction device 10 as well as enter control parameters to control the function of the suction device 10. The computer comprises a control panel 32 and an alphanumeric readout screen 30. The control panel 32 is preferably an alphanumeric keyboard that allows the user to type in control parameters. The readout screen 30 and the control panel 32 are disposed along the front wall 24 of the housing 20. The computer may optionally include a USB port 36 that will allow communication with exterior computers that may store or printout data received from the suction device 10. Furthermore, the suction device 10 may provide wireless communication with exterior personal computers.

The input tube 61 and the output tube 60 are drainage tubes that transport drained fluids from the patient to the fluid collection device 50. The input tube 61 has a fluid receiving end 63 and a housing access end 65. The fluid receiving end 63 of the input tube 61 is secured to the patient (as shown in FIG. 1). The housing access end 65 of the input tube 61 enters into the housing 20 through one of the access holes 29. The input tube 61 drains fluid from the patient and transports it into the housing 20 of the suction device 10. The output tube 60 has a housing access end 64 and a discharge end. The housing access end 64 of the output tube 60 enters into the housing 20 through one of the access holes 29. The discharge end of the output tube 60 is secured to the fluid collection device 50. The output tube 60 transfers drained fluid from the housing 20 to the fluid collection device 50.

The fluid collection device 50 can comprise any form of fluid container. According to the preferred embodiment of the present invention the fluid collection device 50 is a collection bag. The collection bag is mounted to either a stationary or portable stand and receives the drained fluids from the suction device 10. The collection device 50 is preferably positioned below the suction device 10 to allow gravity to assist the flow of fluid. The collection bag shown in FIG. 2 is only an example and any conventional fluid collection device may be used in its place.

FIG. 3 is a front view of the device 10 with the housing 20 broken away to show the interior components of the suction device that are located inside of the housing 20. The suction-producing member 70 is positioned inside of the housing 20.

The suction-producing member 70 is preferably a conventional suction bulb, or a specially designed suction bulb having varying degrees of thickness and stiffness. The suction bulb 70 has a collapsible main body 71. The main body 71 of the suction bulb 70 is flexible and generally ellipsoidal. The main body 71 of the bulb is preferably made from a material such as medical grade polyvinyl chloride or silicone rubber. The material may also be translucent so that the interior of the main body 71 may be viewed. The main body 71 may be made with varying thickness to provide varying degrees of stiffness. The varying stiffness will vary the degree of suction created by the suction bulb 70. The suction bulb 70 also comprises an inlet end 72 having a circular opening. A cap 74 is secured to the top of the bulb 70, covering the circular opening of the inlet end 72. The cap 74 is preferably made from a semi-rigid plastic material. The cap 74 is secured to the inlet end 72 of the bulb 70 by an adhesive, such as cyclohexanone.

Inlet valve 78 and outlet valve 76 are disposed at the top surface of the cap 74. The inlet valve 78 and the outlet valve 76 receive the input tube 61 and the output tube 60, respectively. The inlet valve 78 receives the access end 65 of the input tube 61 after it passes through the access hole 29 in the housing 20. The outlet valve 76 receives the access end 64 of the output tube 60 after it passes through the access hole 29 in the housing 20. The inlet valve 78 and the outlet valve 78 are one-way valves. The inlet valve 78 only allows fluid to pass through the input tube 61 into the suction bulb 70, but does not let fluid leave the suction bulb 70 through the input tube 61. The outlet valve 76 allows fluid to pass from the suction bulb 70 into the output tube 60, but does not allow fluid to enter into the suction bulb 70 from the output tube 60. The arrows in FIG. 3 show the direction of flow of fluid through the suction device 10 as regulated by the inlet valve 78 and the outlet valve 76. The outlet cracking pressure, or pressure required to open the outlet valve 76 for fluid flow, is set greater than the inlet cracking pressure, or pressure required to open the inlet valve 78, in. order to prevent siphoning.

FIG. 3 further depicts an optional position for the inlet valve 78. The inlet valve 78a may be positioned on the bottom edge 73 of the suction bulb 70. The input tube 61a is received by the inlet valve 78a after passing through an access hole in the bottom wall 21 of the housing 20. When the bottom valve 78a is used in place of inlet valve 78, the fluid is drained from the patient and enters into the suction bulb 70 through the bottom edge 73 of the suction bulb 70. Additionally, the direction of fluid flow may be reversed so that the drained fluid enters through the top of the bulb 70 and exits out of the bottom of the bulb 70.

The suction device 10 further includes a compressor assembly for continuously compressing the suction bulb 70. The compressor assembly comprises a compression device 80 and a sensor 82. The compression device 80, as shown in FIG. 3, is preferably a stepper motor operated screw piston. The compression device 80 forcibly engages the main body 71 of the suction bulb 70. The compression device 80 impacts the main body 71 of the suction bulb 70 to compress the bulb 70, forcing liquid and gas through the outlet valve 76 and outlet tube 60 into the collection device 50. A sensor 68 measures the relative amounts of liquid and gas passing through output line 60 and sends a signal to the computer to place in memory and make the value available for display on screen 30.

Once the suction bulb 70 is fully compressed the compression device 80 releases from the main body 71 and the suction bulb 70 decompresses spontaneously by elastic recoil of the suction bulb 70. During recoil the suction bulb 70 draws additional fluid and gas through the inlet valve 78. The sensor 82 detects when the suction bulb 70 is spontaneously decompressed by elastic recoil. The sensor 82 sends signals to the computer, which controls the movement of the compression device 80. The compression device 80 then impacts the main body 71 of the suction bulb 70, again compressing the bulb 70.

The user of the suction device 10 will secure the input tube 61 to the patient and the output tube 60 to the fluid collection device 50. The user then enters in the control parameters through the control panel 32. The compression device 80 will then compress the suction bulb 70. Once the suction bulb 70 is fully compressed the sensor 82 will notify the computer to retract the compression device 80. The main body 71 of the suction bulb 70 will then decompress spontaneously by elastic recoil. As the main body 71 decompresses, the suction device 70 applies a gentle suction to the input tube 61. The gentle suction facilitates fluid drainage from the patient along with any air present. Once the suction bulb 70 is completely filled with liquid and air, the sensor 82 will send a signal to the computer. The computer will then activate the compression device 80 to recompress the suction bulb 70, emptying the contents of the suction bulb 70 into the fluid collection device 50 through the output tube 60. Once the suction bulb 70 is empty, the sensor 82 will notify the computer to retract the compression device 80 so the main body 71 of the suction bulb 70 will decompress spontaneously by elastic recoil. Once the main body 71 of the suction bulb 70 is decompressed the entire process will automatically restart.

The computer will record in memory the timing of each compression/decompression event. The user will be able to enter parameters, the satisfaction of which will notify the user that each of the events has occurred. The readout screen 30 will display the data from the computer as well as alert the user of excessive fluid or air production. The computer also functions to calibrate the volume of fluid/air for each compression; to allow delay between compression/recoil cycles; and to determine the percentage of the fluid-to-air ratio by stiffness of the bulb, by an electronic sensor in the fluid line, or by any other method. All compression/decompression events are recorded, along with their timeline, for future use and manipulation. The ratio of fluid to volume is recorded with their timeline. Selected “alarm values” are set by the operator through the alphanumeric keyboard to notify the operator by sound signal and/or screen readout of excessive or insufficient air or fluid production.

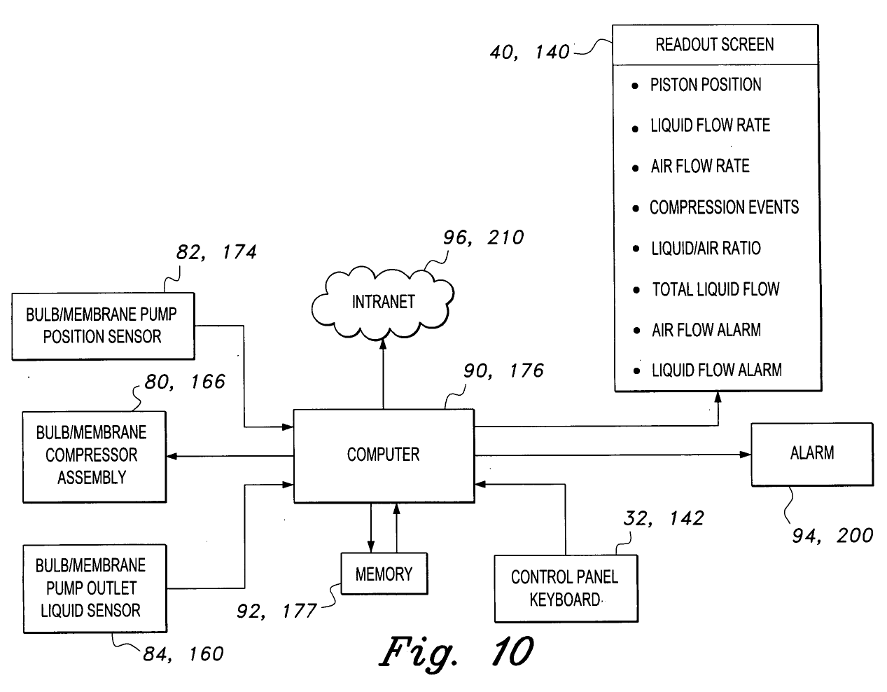

Referring to FIG. 10, there is shown a diagram of the computer control and recording system, wherein an operator enters desired parameters, such as alarm values and selected length of compression stroke of the bulb compressor assembly, and activates the operation of the suction system 10. The suction bulb 70 is first evacuated by the computer 90 signaling the bulb compressor assembly 80 to advance the piston against the suction bulb 70 to force air therefrom through the output tube 60. The distance of the advance of the piston may be set by the operator at control panel 30 to be controlled by the computer 90. The piston is then returned to its withdrawn position, allowing suction bulb 70 to apply suction through inlet 61 to fill with liquid and gas therefrom.

When suction bulb 70 returns to its decompressed state by elastic recoil, the wall of the bulb 70 trips the sensor 82, which signals the computer 90 to again advance the piston of bulb compressor assembly 80. During the elastic recoil, liquid and air are drawn into the suction bulb 70 through inlet 61. As the piston is advanced, the outlet liquid sensor 84 signals the computer 90 when liquid is flowing through the outlet 60, the signal being interrupted when air is flowing therethrough. The total volume of liquid and air expelled during the stroke of the piston is known based on calibration of the system employing a particular size bulb, the selected length of piston stroke as entered by the operator, and the output of the liquid/gas sensor 68.

From this information, the computer may calculate parameters including overall liquid flow rate and air flow rate during the operation of the suction system 10 with the piston continuing to cycle between its expulsion stroke and its return stroke, the liquid flow rate and air flow rate being recorded by the computer in memory 92 over time. The computer may also calculate total flow of liquid and of air by integrating their flow rates over time of operation of the suction system 10. The computer may also calculate the average ratio of liquid to air flow and store this in memory 92. The computer may also compare the parameters including the ratio of liquid-to-air flow, the air flow rate, the liquid flow rate, total liquid flow, and total air flow with maximum and/or minimum alarm values entered into the computer 10 by the operator, and when the values of those parameters are reached, the computer may send a signal to activate alarm 94, which may emit a sound or display a light. The alarm signal may also be sent to the readout screen 30 and to remote monitoring devices 96 by means of intranet 96.

The screen readout may display a particular parameter such as piston position as a function of time, liquid flow rate, air flow rate, number of compression events as a function of time, liquid/air ratio, total liquid flow, total air flow, and alarm signals upon selection by the operator at the control, or all of the parameters may be shown simultaneously as desired. All parameters may be directed from computer 90 or memory 92 to a printer within intranet 96. The intranet may be connected with a plurality of suction devices 10, enabling a single operator to monitor a number of patients simultaneously.

Currently used mechanical suction devices are subject to failure or human error resulting in harm and potential death to the patient. The one-way valves of the suction device 10 prevent harm to the patient caused by leakage by preventing fluid from going back to the patient's body. The integrated computer provides many advantages, including automating the fluid drainage, as well as improving medical record keeping. The wireless communication port 36 will allow multiple patients to be monitored at the same time for such parameters as fluid and air production from a central location. Finally, the suction device 10 is small enough that it is fully portable. The suction device 10 further comprises a battery 40 (shown in FIGS. 2 and 3), which will power the suction device 10 when it is used in transport or in the home of a patient.

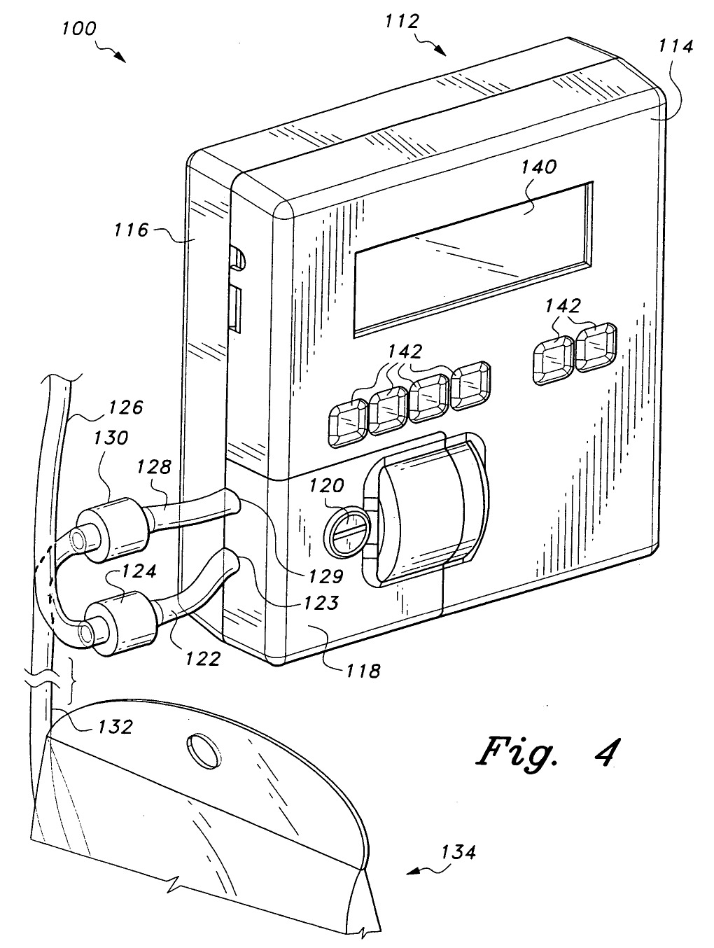

Referring to FIG. 4, another embodiment of the continuous safe suction device of the present invention is shown, designated generally as 100. Suction device 100 has a split casing 112 including a front cover 114 and a rear casing 116. Front cover 114 includes a suction storage member access cover 118 removably held in place by access cover securing screw 120. Input line 122 enters casing 112 through casing inlet 123 in front cover 114. A one-way valve 124 connects input line 122 to patient fluid drainage line 126. Output line 128 exits casing 112 through casing outlet 129 in front cover 114. A one-way valve 130 connects output line 132 for drainage into fluid collector 134.

Casing 112 has an alphanumeric readout screen 140 and alphanumeric control keys 142 extending through the front cover 114 for operation and monitoring of the suction device 100.

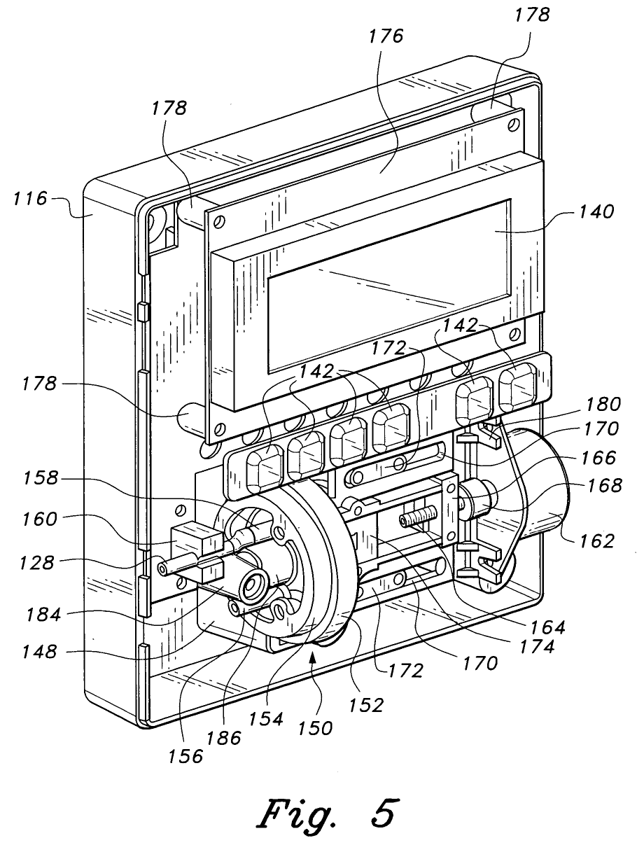

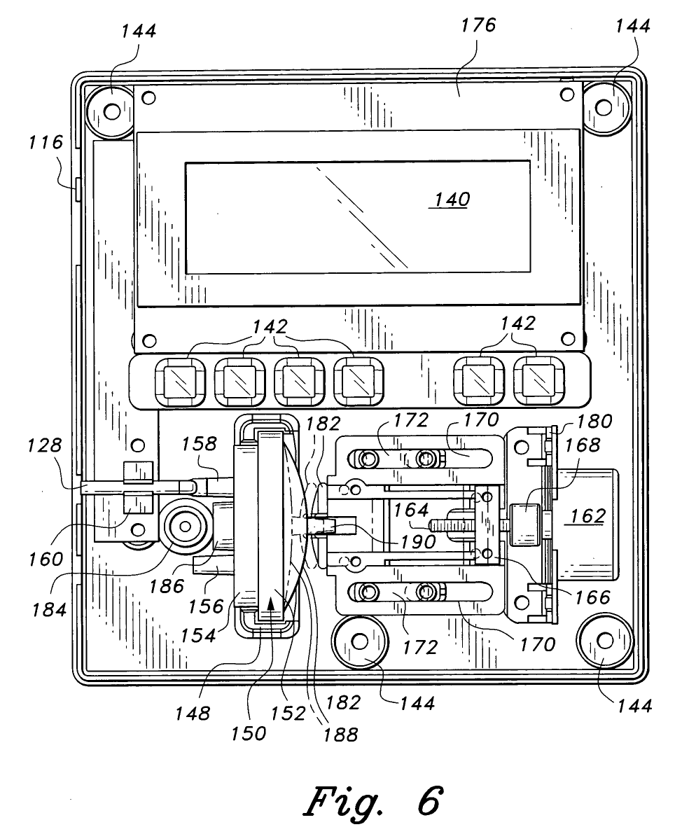

Referring to FIGS. 5 and 6, there are shown a perspective view and a front elevation view, respectively, of the suction device 100 (see FIG. 4) with the front cover 114 removed. Alphanumeric readout screen 140 and alphanumeric control keys 142 are mounted to and electrically connected with computer and memory board 176, mounted to rear casing 116 by computer and memory board spacer supports 178. Membrane suction storage member holder 148 is connected to rear casing 116 and is semicircular in shape and, together with suction storage member access cover 118, supports membrane suction storage member 150. Membrane suction storage member 150 includes membrane holder ring 152 connected with membrane suction storage member base 154 to secure flexible membrane 188. Membrane suction storage member 150 has an input nozzle 156 and an output nozzle 158 formed by suction storage member base 154 to which input line 126 (omitted from FIGS. 5 and 6 for clarity, but shown in FIG. 4) and output line 128 are respectively connected during operation of suction storage member 150.

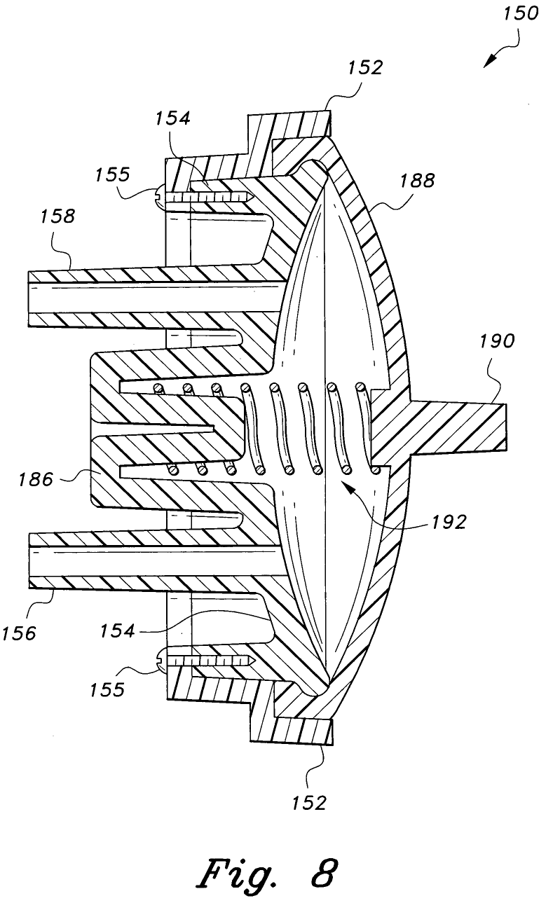

Liquid/gas sensor 160 is mounted so as to envelop output line 128 and is electrically connected with computer 176. Stepper motor 162 is electrically connected with computer 176 and rotates screw drive 164 in a clockwise or counterclockwise direction, depending on signals from computer 176. Screw drive 164 drives frame piston 166 at its rear end, the stroke being adjustable by stroke adjuster 168. Frame piston 166 is supported in piston travel slots 170 by travel supports 172 fastened to rear casing 116. Optical sensor 174 is mounted on the forward end portion of frame piston 172 and detects the position of membrane sensor target 190 extending from the center of flexible membrane 188. Stepper motor 162 is mounted to rear casing 116 by stepper motor mount 180. Membrane engaging pads 182 are mounted at the forward end of frame piston 172 and extend from above and below membrane sensor target 190 to engage flexible membrane 188. Access cover screw receiver 184 extends forward to receive access cover screw 120 for mounting suction storage member access cover 118. Membrane suction storage member base 154 includes an axially located spring receiver 186 holding membrane resilience spring 192 (see FIG. 8).

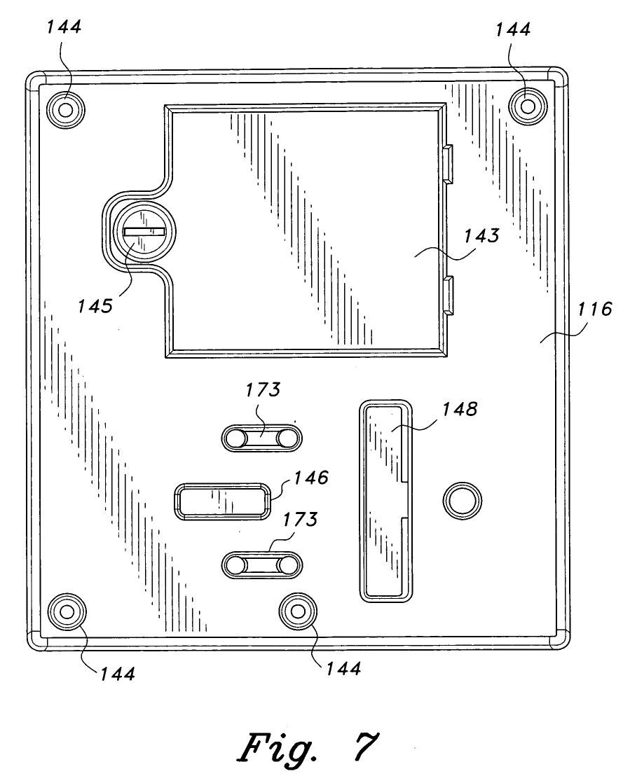

Referring to FIG. 7 there is shown a rear elevation view of the rear casing 116 having split casing securing mounts 144 for computer and memory board spacer supports 178. Also shown is a battery access cover 143 removably secured by battery access cover securing screw 145. Travel support access ports 173 support frame piston travel supports 172. Data access port 146 allows connection with an intranet. A wireless communication port is provided (not shown) similar to port 36 of the first embodiment (see FIG. 2) to allow multiple patients to be monitored from a central location, simultaneously, for parameters such as fluid and air production.

Referring to FIG. 8, there is shown a section view of a design for a membrane suction storage member 150 similar to that of FIGS. 5 and 6, wherein flexible membrane 188 is held around its periphery by membrane holder ring 152 and membrane suction storage member base 154 as held by attachment screws 155. As is seen, suction storage member base spring receiver 186 holds and axially locates membrane resilience spring 192, which provides spring pressure against membrane 188 at the inner side of membrane sensor target 190. Input nozzle 156 and output nozzle 158 are formed by, and extend from, base 154 for connection with input and output lines 122 and 128, respectively. By removal of attachment screws 155, base 154 may be separated from membrane holder ring 152 and another spring 192 of greater or less stiffness may be substituted to adjust the suction of the membrane suction storage member 150.

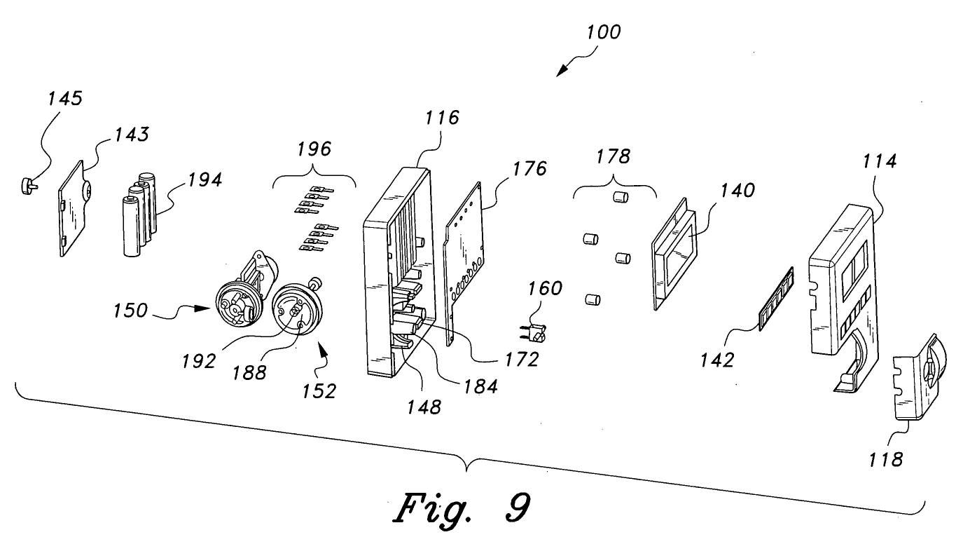

Referring to FIG. 9, there is shown an exploded view of the continuous safe suction device embodiment 100 showing suction storage member access cover 118, front cover 114, alphanumeric control keys 142, alphanumeric readout screen 140, computer and memory board 176 and spacer supports 178, liquid sensor 160, rear casing 116 having membrane suction storage member holder 148 and access cover screw receiver 184, membrane suction storage member 150, membrane holder ring 152 with membrane 188 and membrane resilience spring 192, battery access cover 143 with securing screw 145, and batteries 194 with battery to computer connectors 196.

Referring to FIG. 10 there is a diagrammatic representation of the computer-controlled operation of the inventive bulb suction devices 10, 100. In the operation of the first embodiment 10, the sensor 82 senses when the bulb is fully extended and ready for emptying at which time it is sends a signal to the computer 90 which sends a signal to the bulb compressor assembly 80 to extend against the bulb wall to evacuate its contents of liquid and air. Once the full stroke is completed, the bulb compressor assembly 80 retracts to a point set by the control panel keyboard for another suction filling step.

During the evacuation of the bulb, the outlet liquid/gas sensor 84 sends data to computer 90 which calculates instantaneous and average liquid/gas ratio employing memory 92 and displays this value upon the readout screen 30 upon request K at the control panel keyboard 32. Upper and lower alarm values of liquid/gas ratio may be set by the control panel keyboard 32 in computer 90 so as to activate alarm 94 (sound and/or light) when calculated values are outside the set values. These calculated values and alarm signal may also be sent over a hard wired or radio signal to a central operator station (not shown).

Other parameter values may be calculated by computer 90, using memory 92 and displayed either constantly or upon command by the control panel keyboard 32, including piston position, instantaneous and average liquid flow rate and air flow rate, a total of the number of compression events from a set beginning time, the total liquid flow from a set beginning time, the total air flow from a set beginning time, a visual air flow alarm corresponding to alarm 94 (sound and/or light), and a visual liquid flow alarm. The control panel keyboard 32 may be reset to time zero when desired. The details of the computer hardware and software are not described as the design and construction of such a system and software are well known in the art of computer-controlled instruments.

As further illustrated in FIG. 10, the operation of the embodiment 100 of the inventive membrane suction storage member suction device is also illustrated. The operation of the embodiment 100 is similar to that of that of embodiment 10 described above with the exception of the interaction between the membrane position sensor 174, the membrane compressor assembly 166, and the computer 176 and memory 177. As best seen in FIG. 6, the membrane position sensor 174, which is preferably an optical sensor, detects the position of the cone-shaped membrane sensor target 190. When the membrane 188 extends from a compressed configuration (shown by hidden lines) during suction a desired incremental distance, the position sensor 174 signals the computer 176 to back off frame piston 166 a selected incremental distance. The membrane resilience spring 192 is then able to extend the membrane outward incrementally as suction caused thereby pulls in liquid and air from the patient P.

Once the membrane, as indicated by membrane sensor target 190, is detected by position sensor 174, a signal is sent to the computer to back off frame piston 166 of compressor assembly 80 another increment as set by control panel keyboard 142. The withdrawal distance and time of each increment may be controlled by the computer through input values entered by the operator at the alphanumeric control keyboard. This incremental membrane expansion and compressor assembly back-off, as controlled by computer 176, continues until the maximum expansion of membrane 188 is reached at which time the computer signals the compressor assembly to fully engage membrane 188 and the piston thereof is extended to empty the now full membrane suction storage member 150. At this time the membrane suction storage member outlet liquid sensor 160 sends data to the computer 176, which stores data in memory 177 and calculates, along with data from the compressor assembly 166, the values for display on readout screen 140 at the request of the operator at control panel keyboard 142.

All the values of the readout screen 40 of the bulb suction embodiment 10 are available on readout screen 140, including piston position as a function of time during its incremental back-off stroke and its full extension-emptying stroke. Alarm values set by control keyboard panel 142 may be shown and monitored on readout screen 140 and the computer may activate alarm 200.

The liquid sensor is commercially available as model OPB350 from OPTECK Technology, inc., Carrolton, Tex.

It is to be understood that the present invention is not limited to the embodiments described above, but encompasses any and all embodiments within the scope of the following claims.

1. A continuous safe suction device for draining liquid and trapped air from a patient, comprising:
a housing defining at least one input tube opening and at least one output tube opening;
a recoil suction storage member having an elastic wall having a compressed position and a fully recoiled position;
an input tube extending through said input tube opening and fluidly connected with said suction storage member and adapted for connection with the patient;
an output tube extending through said output tube opening and fluidly connected with said suction storage member;
a first one-way valve having a first cracking pressure and located in line with said input tube for preventing backflow from said suction storage member;
a second one-way valve in said output tube having a second cracking pressure greater than the cracking pressure of said first one-way valve for preventing entry of air or liquid into said suction storage member and maintaining fluid and air collected within said suction storage member to a pressure below said second valve cracking pressure; and
a compressor assembly periodically engaging and compressing said suction storage member, thereby raising the pressure of said liquid and air within said suction storage member above said second valve cracking pressure, thereby evacuating liquid and gas through said output tube and said second one-way valve.

2. The continuous safe suction device of claim 1, wherein said compressor assembly comprises a retractable piston for engaging said elastic wall and compressing said suction storage member.

3. The continuous safe suction device of claim 2, further comprising a sensor positioned to detect the position of said elastic wall, at least upon said elastic wall completing recoil to said fully recoiled position, and a computer electrically connected with said sensor and said compressor assembly, said sensor signaling said computer upon detection of said elastic wall at said fully recoiled position, said computer controlling said compressor assembly to cause said piston to engage said elastic wall, compressing said suction storage member, and to retract to a withdrawn position, thereby emptying said suction storage member of liquid and air.

4. The continuous safe suction device of claim 3, wherein said computer has a control panel mounted on said housing and electrically connected to said computer for controlling the length of stroke of said piston and, thereby, the degree of compression of said suction storage member.

5. The continuous safe suction device of claim 4, further comprising a liquid sensor arranged around said output tube, said liquid sensor being electrically connected with said computer, said liquid sensor sending a signal to said computer upon passage of liquid through said output tube.

6. The continuous safe suction device of claim 5, further comprising a readout screen electrically connected with said computer, said computer calculating parameters including liquid flow rate, air flow rate, liquid/air ratio, total liquid flow and total air flow through said output tube, said readout screen displaying selected said parameters upon signal provided a control panel operator.

7. The continuous safe suction device of claim 6, further comprising an intranet connection located on said housing and electrically connected with said computer for providing signals to remote readout screens and printers corresponding to said parameters.

8. The continuous safe suction device of claim 7, further comprising an alarm electrically connected with said computer, said control panel, by an operator, providing said computer with ranges of parameter values such as to activate said alarm upon any of said parameters calculated by said computer falling outside said respective range of parameter values.

9. The continuous safe suction device of claim 7, wherein said compressor assembly provides a signal to said computer indicating piston position, said computer providing signals to said readout screen indicating piston position as a function of time and the number of compression events and their occurrence as a function of time.

10. The continuous safe suction device of claim 10, wherein said suction storage member is a recoil suction bulb, and wherein said compressor assembly has a piston operated by a screw and stepper motor.

11. The continuous safe suction device of claim 10, wherein said flexible wall of said suction storage member is a membrane, said suction storage member having a body having an input nozzle and an output nozzle, said membrane being removably sealed with said body.

12. The continuous safe suction device of claim 11, wherein said suction storage member has a membrane resilience spring therein under compression and located between said body and said membrane.

13. The continuous safe suction device of claim 12, wherein said membrane has a central portion having a generally cone-shaped sensor target extending- outward therefrom toward said piston of said compressor assembly, said membrane resilience spring meeting said membrane at said central portion thereof.

14. The continuous safe suction device of claim 13, wherein said elastic wall position sensor comprises an optical sensor located on said piston and overlapping said cone-shaped sensor target.

15. The continuous safe suction device of claim 14, wherein said piston is withdrawn incrementally by said compressor assembly in response to said computer signal given in response to said optical sensor detecting said cone-shaped sensor target and signaling said computer, whereby said resilience spring gradually forces said membrane outward as said recoil suction storage member fills with liquid and air from said patient.

16. The continuous safe suction device of claim 15, wherein said piston has membrane engaging pads and said piston is withdrawn in increments of displacement and time by said computer in response to values input into said computer through said control panel.

17. The continuous safe suction device of claim 16, wherein said piston is a frame piston having upper and lower travel slots and said housing has upper and lower piston travel supports attached thereto and extending respectively into said upper and lower travel slots.

18. The continuous safe suction device of claim 17, wherein said frame piston is operated by a stepper motor.

19. The continuous safe suction device of claim 18, wherein said housing is a split casing having a rear casing and a removable front cover, said front cover comprising a separately removable suction storage member access cover, said split casing and said removable suction storage member access cover supporting said recoil suction storage member having said flexible membrane, said computer having a memory and mounted on a computer and memory board mounted to said rear casing, and said readout screen.

20. A continuous safe suction device for draining liquid and trapped air from a patient, comprising:
a housing defining at least one input tube opening and at least one output tube opening;
a recoil suction storage member having an elastic wall having a compressed position and a fully recoiled position;
an input tube extending through said input tube opening and fluidly connected with said patient and said suction storage member;
an output tube extending through said output tube opening and fluidly connected with said suction storage member;
a first one-way valve having a first cracking pressure and located in line with said input tube for preventing backflow from said suction storage member;
a second one-way valve in said output tube having a second cracking pressure greater than the cracking pressure of said first one-way valve for preventing entry of air or liquid into said suction storage member and maintaining fluid and air collected within said suction storage member to a pressure below said second valve cracking pressure;
a compressor assembly periodically engaging and compressing said suction storage member, thereby raising the pressure of said liquid and air within said suction storage member above said second valve cracking pressure, thereby evacuating liquid and gas through said output tube and said second one-way valve; and
an electric power source for driving said compressor assembly;
said compressor assembly comprising a retractable piston for engaging said elastic wall and compressing said suction storage member;
said safe suction device further comprising a sensor so arranged as to detect the position of said elastic wall, at least upon said elastic wall completing recoil to said fully recoiled position, and a computer electrically connected with said sensor and said compressor assembly, said sensor signaling said computer upon detection of said elastic wall at said fully recoiled position, said computer controlling said compressor assembly so as to cause said piston to engage said elastic wall, compressing said suction storage member, and retract to a withdrawn position, thereby emptying said suction storage member of liquid and air;
said computer having a control panel mounted on said housing and electrically connected to said computer for controlling the length of stroke of said piston and, thereby, the degree of compression of said suction storage member;
said safe suction device further comprising a liquid sensor arranged around said output tube, said liquid sensor being electrically connected with said computer, said liquid sensor sending a signal to said computer upon passage of liquid through said output tube;
said safe suction device further comprising a readout screen electrically connected with said computer, said computer calculating parameters including liquid flow rate, air flow rate, liquid/air ratio, total liquid flow and total air flow through said output tube, said readout screen displaying selected said parameters upon signal provided a control panel operator;
said safe suction device further comprising an intranet connection located on said housing and electrically connected with said computer for providing signals to remote readout screens and printers corresponding to said parameters;
said safe suction device further comprising an alarm electrically connected with said computer, said control panel, by an operator, providing said computer with ranges of parameter values such as to activate said alarm upon any of said parameters calculated by said computer falling outside said respective range of parameter values;
said compressor assembly providing a signal to said computer indicating piston position, said computer providing signals to said readout screen indicating piston position as a function of time and the number of compression events and their occurrence as a function of time;
said flexible wall of said suction storage member being a membrane, said suction storage member having a body having an input nozzle and an output nozzle, said membrane being removably sealed with said body;
said suction storage member having a membrane resilience spring therein under compression and located between said body and said membrane;
said membrane having a central portion having a generally cone-shaped sensor target extending outward therefrom toward said piston of said compressor assembly, said membrane resilience spring meeting said membrane at said central portion thereof;
said elastic wall position sensor being an optical sensor located on said piston and overlapping said cone-shaped sensor target; and
said piston being withdrawn incrementally by said compressor assembly in response to said computer signal given in response to said optical sensor detecting said cone-shaped sensor target and signaling said computer;
whereby said resilience spring gradually forces said membrane outward as said recoil suction storage member fills with liquid and air from said patient.
